(19) United States
(12) Patent Application Publication
(10) Pub. No.: US 2007/0059319 A1
(43) Pub. Date: Mar. 15, 2007

(54) Methods of screening for immuno-adjuvants and vaccines comprising anti-microtubule immuno-adjuvants

(75) Inventors: Peter Carlson, Chevy Chase, MD (US); Alexei Miagkov, Columbia, MD (US); Martha Widra, Linthicum, MD (US)

Correspondence Address: FOLEY AND LARDNER LLP;SUITE 500, 3000 K STREET NW, WASHINGTON, DC, 20007, US

(73) Assignee: Caliper Life Sciences, Inc.

(21) Appl. No.: 11/518,522

(22) Filed: Sep. 11, 2006

Related U.S. Application Data
(60) Provisional application No. 60/717,022, filed on Sep. 15, 2005.
(60) Provisional application No. 60/763,368, filed on Jan. 31, 2006.

(51) Int. Cl. A61K 39/00; C12Q 1/00; A61K 31/165; A61K 31/4745

(52) U.S. Cl. 424/185.1; 435/4; 514/283; 514/620

(57) ABSTRACT

A method of screening for agents that stimulate the innate immune system in mammals employs markers that respond to Toll-like receptor binding. Agents identified in the assay boost both innate and adaptive immune responses, when administered alone or in combination with vaccines.

### CROSS-REFERENCE OF RELATED APPLICATION

This application claims priority from Provisional Application U.S. Application 60/717,022, filed Sep. 15, 2005, incorporated herein by reference in its entirety. This application also claims priority from Provisional Application U.S. Application 60/763,368, filed Jan. 31, 2006, incorporated herein by reference in its entirety.

### BACKGROUND OF THE INVENTION

A significant development in the field of human immunology has been the recognition that our immune system comprises two arms that perform distinct yet complementary roles: the innate immune system and the adaptive immune system. The innate immune system provides rapid, nonspecific and generalized defense mechanisms, implemented by cells and molecules that are active against a wide range of potential pathogenic microorganisms. Key elements of the innate immune system include macrophages and granulocytes, both of which are capable of phagocytosis (engulfing of foreign particles or antigens), and natural killer (NK) cells.

The innate immune system does not play a direct role in the development of specific immunity or immunological “memory.” These are hallmarks of the adaptive immune system. Nevertheless, the innate immune system does impact the development of specific immunity and immunological memory by activating a signaling system that stimulates lymphocytes (B- and T-cells). Lymphocytes are primary actors in the adaptive immune system. Activated B-cells can mature into antibody-producing factories. Activated T-cells can become assassins that directly kill diseased cells or can become messengers that activate other elements in the immune system.

Accordingly, agents that stimulate the innate immune system not only stimulate protective activities of the innate immune system, but also can promote and sustain B- and T-cell responses of the adaptive immune system. Such agents can be used as adjuvants in vaccines.

The practice of immunizing mammals, especially humans, with vaccines is common. Considerable effort has been, and is being, made to extend this practice to cover an extensive array of diseases. One problem frequently encountered in the course of immunization, however, is vaccine antigens that are not sufficiently immunogenic to raise a sufficiently high antibody titer, i.e., an antibody titer sufficiently high to protect against subsequent challenge or to maintain the potential for mounting a sufficient response over extended time periods. Another problem is that vaccines may be deficient at inducing cell-mediated immunity, which is a primary immune defense against bacterial and viral infection.

To obtain a stronger humoral and/or cellular response, it is common to include an adjuvant (immunopotentiator) in vaccine formulations. Adjuvants that previously have been used to enhance an immune responses include aluminum compounds (all generally referred to as “alum”), oil-in-water emulsions (often containing other compounds), complete Freund's adjuvant (CFA, an oil-in-water emulsion containing dried, heat-killed Mycobacterium tuberculosis organisms), pertussis adjuvant (a saline suspension of killed Bordatella pertussis organisms), and saponins.

The mechanisms by which adjuvants function are poorly understood, and whether or not a particular adjuvant will be sufficiently effective in a given instance is not predictable. There remains a need in the art for additional effective adjuvants, particularly adjuvants that stimulate both innate immunity and adaptive immunity.

### SUMMARY OF THE INVENTION

To address this and other needs, the present invention provides methods of screening for agents that stimulate the innate immune system in mammals, methods of stimulating the innate immune system, and vaccines comprising agents that stimulate the innate immune system.

In one aspect, the invention provides a method of screening for agents that stimulate the innate immune system in a mammal. This method includes bringing a candidate agent into contact with a cellular component of the innate immune system. The cellular component can then be tested to determine whether contact with the candidate agent induces changes in the levels of cellular markers that are associated with stimulation of the innate immune system. The levels of these markers can then be correlated with a probability that the candidate agent stimulates the innate immune system.

In another aspect, the invention provides a method of stimulating the innate immune system in mammal by administering to that mammal a vaccine and a microtubule depolymerizing agent.

In still another aspect, the invention provides a method of stimulating the innate immune system in a mammal by administering a microtubule depolymerizing agent to the mammal. The mammal is selected to be one that is in need of increased innate immunity, but which does not have a cell proliferative disorder.

In yet another aspect, the invention provides vaccine that comprises a microtubule depolymerizing agent.

### BRIEF DESCRIPTION OF THE DRAWINGS

FIG. 1 shows results of an RT-PCR analysis evaluating the expression of TLRs on three cell lines.

FIG. 2 shows results of a flow cytometry analysis evaluating how TLR ligand binding affects the expression of cell surface molecules on THP-1 cells.

FIG. 3 shows results of assays evaluating how TLR ligand binding affects the expression of cytokines by THP-1 cells.

FIG. 4 shows results of assays evaluating how TLR ligand binding affects the expression of two cell surface markers and three cytokines by THP-1 cells; the markers represent a panel for use in 5-plex high throughput screening.

FIG. 5 shows results indicating the sensitivity of assays to changes in cytokine expression after TLR ligand binding.

FIG. 6 shows results indicating the sensitivity of assays to changes in co-stimulatory molecule expression after TLR ligand binding.

FIG. 7 shows results from an evaluation of assay reproducibility for co-stimulatory molecules.

FIG. 8 shows results from an evaluation of assay reproducibility for cytokine results.

### DETAILED DESCRIPTION OF THE PREFERRED EMBODIMENTS

The present inventors have discovered a method of screening for agents that stimulate the innate immune system, methods that employ such agents to stimulate the innate immune system, and vaccines that comprise such agents.

The innate immune system is that portion of the broader immune system that provides rapid, nonspecific and generalized defense mechanisms. This portion of the immune system detects constitutive and conserved products of microbial metabolism. Microbes have many metabolic pathways and gene products that are not found in mammalian cells. A number of these pathways perform housekeeping functions, and their products are conserved among microorganisms in the same class. Exemplary proteins made by bacteria, but not eukaryotic cells, include lipopolysaccharide (LPS) lipoproteins, peptidoglycan and lipoteichoic acids (LTAs). The recognition of such proteins in a mammal can signal a bacterial infection. Target proteins are not necessarily identical in every microorganism, but target proteins generally have conserved molecular patterns across microorganisms. These patterns are called pathogen-associated molecular patterns (PAMPs).

Receptors of the innate immune system that recognize PAMPs are called pattern-recognition receptors (PRRs). A major group of PRRs is the family of Toll-like receptors (TLRs). TLRs are a family of type I transmembrane receptors characterized by an extracellular leucine-rich repeat (LRR) domain and an intracellular Toll/IL-1 receptor (TIR) domain. TLR signaling can induce the production of proinflammatory cytokines and upregulate expression of costimulatory molecules. This activates not only innate immunity, but also adaptive immunity.

In the inventive methods of screening for agents that stimulate the innate immune system, a candidate agent is brought into contact with a cellular component of the immune system. The cellular component may be any cell that expresses a pattern-recognition receptor (PRR). Preferably, the PRR is a Toll-like receptor, such as TLR-1, TLR-2, TLR-3, TLR-4, TLR-5, TLR-6, TLR-7, TLR-8, TLR-9 or TLR-10. The PRR also could be a cytokine receptor or a NOD protein (i.e., a protein having a nucleotide-binding oligomerization domain).

Exemplary cellular components that express a PRR are monocytes, dendritic cells, macrophages, natural killer (NK) cells, and B-cells. Monocytes are preferred cellular components. The cellular component may be a cell line. Some exemplary cell lines are THP-1, HL-60, RPMI-8228, PBMC, KG-1, Ramos, BMDC, TF-1a, and HEK-TLR9. Among these, THP-1 is a preferred cell line.

The inventive screening methods further comprise testing the cellular component for one or more markers associated with stimulation of the innate immune system. The markers include any molecule that experiences a measurable qualitative or quantitative change as a result of a ligand/agent binding to a PRR. For example, ligand binding to a PRR may change the expression of cytokines, chemokines, co-stimulatory molecules or antigen presenting molecules of the major histocompatibility complex. The measurable change most commonly is an increase or decrease in the quantity of marker. Preferably, more than one marker is tested (i.e., a panel of markers), which provides a more complete view of how ligand binding impacts the innate immune system. For example, two, three, four, five, six, seven, eight, nine, ten or even more markers may be multiplexed to provide an assay that yields information about how ligand binding to a PRR impacts multiple aspects of the innate immune system.

In the case of Toll-like receptors, ligand binding can cause increased cellular expression of MHC class I molecules (e.g., HLA-A, HLA-B or HLA-C), MHC class II molecules (e.g., HLA-DR, HLA-DQ, HLA-DP), co-stimulatory molecules (e.g., CD80 (B7-1), CD86 (B7-2), CD40, CD54 (ICAM-1)), and/or cytokines (TNF-α, IL-8, IL-6, MCP-1, MIP-1 α, MIP-1β, RANTES, IP-10, MIG).

The selection of one or more markers for testing is a matter of routine skill, and depends in large part of the cellular component being used. Different types of cells and even cells of the same type derived from different cell lines may vary in their expression of pattern-recognition receptors. Additionally, cells expressing the same PRR may respond differently to ligand binding to the PRR. Assays for determining whether a given cell expresses a particular PRR and for measuring whether a particular molecule can function as a marker of ligand binding to a PRR are well known in the art.

In THP-1 monocytes, the surface markers MHC class I, CD80, CD40, CD54, and CD86 become upregulated when ligands bind to TLRs. MHC class II is not upregulated by such binding, but could be upregulated in another type of cell, such as a dendritic cell or macrophage, or another monocyte cell line. In THP-1 monocytes, the cytokines TNF-α, IL-8, IL-6, MCP-1, MIP-1 α, MIP-1β, RANTES, IP-10 and/or MIG become upregulated when ligands bind to TLRs. Again, the cytokine profile for another type of cell or another monocyte cell line could differ.

A preferred embodiment of the invention employs THP-1 monocyte cells as the cellular component of the innate immune system and employs CD80, CD54, TNF-α, IL-8, and RANTES as markers associated with stimulation of the innate immune system.

The inventive screening methods further comprise correlating the level of tested markers with a probability that a candidate agent stimulates the innate immune system. Changes in a single marker or combination of markers can indicate stimulation of the innate immune system, depending on the cellular component and markers under evaluation. Likewise, changes in certain markers could indicate suppression of the innate immune system. The skilled artisan will appreciate the impact that each marker under evaluation could have on the innate immune system, and will be able to interpret the results of each marker in context.

Screening methods of the invention can be applied to cellular components from the innate immune system of any mammal. Examples of preferred mammals include domestic mammals kept for purposes of food production (e.g., cows, pigs, sheep, goats, rabbits), labor (e.g., horses), companionship (e.g., dogs and cats), research (e.g., rats and mice), and primates. Humans are especially preferred.

In another aspect, the invention provides a method of stimulating the innate immune system in a mammal, such as one of the mammals identified above. The method comprises administering a vaccine and an anti-microtubule agent to the mammal.

In the context of this invention, a vaccine refers to any pharmaceutical composition containing an antigenic molecule or a component that induces the expression of an antigenic molecule in vivo. Vaccines are administered to animals for the purpose of stimulating an immune response to a disease element.

The present inventors have discovered that anti-microtubule agents, such as microtubule depolymerizing agents, can act as adjuvants (immunopotentiators). In this context, anti-microtubule agents refer to any agent that interferes with normal microtubule activity. Such agents stimulate the innate immune system and facilitate the development of acquired immunity by the adaptive immune system, as previously described.

One class of anti-microtubule agents useful in the invention is vinca alkaloids. These are nitrogenous base compounds derived from the pink periwinkle plant, Catharanthus roseus. These compounds have a dimeric asymmetric structure composed of a dihydroindole nucleus (vindoline) linked by a carbon-carbon bond to an indole nucleus (catharanthine). Exemplary vinca alkaloids are vincristine, vinblastine, vindesine, and vinorelbine.

Another class of anti-microtubule agents is taxanes. The prototype taxane is paclitaxel, which initially was isolated from the bark of the Pacific yew, Taxus brevifolia. Another taxane is docetaxel.

Other anti-microtubule agents also are known and encompassed by the present invention. These include colchicines, demecolcine and estramustine.

Anti-microtubule agents may constitute a component of the vaccine formulation administered to a mammal. Alternatively, anti-microtubule agents may be administered prior to the vaccine, subsequent to the vaccine or concurrently with the vaccine, but as part of a separate formulation. A combination of these schedules also may be used. The particular schedule of administration may vary according the particular recipient/patient, vaccine, disease element, and anti-microtubule agent. Ideally, the anti-microtubule agent will be administered on a schedule and at a dosage that effectively stimulates the innate immune system without causing toxicity. Determining an appropriate schedule and dosage can readily be performed by those skilled in the art.

In another aspect, the invention provides a method of stimulating the innate immune system in a mammal by administering an anti-microtubule agent to a mammal that does not have a cell proliferative disorder. In this context, a cell proliferative disorder is a disease condition characterized by excessive cell growth. Cancer is a prime example of such a cell proliferative disorder.

In still another aspect, the invention provides a vaccine that comprises an anti-microtubule agent as an adjuvant. The anti-microtubule agent may be any of those previously described.

The vaccines also comprise an antigenic molecule or a component that induces the expression of an antigenic molecule in vivo. The antigenic molecule or component that induces the expression of an antigenic molecule is selected for the purpose of stimulating an immune response to a disease element.

In the context of the present invention, antigens are molecules capable of initiating a humoral or cellular immune response in a recipient of the antigen. Antigens preferably are elements of a disease for which vaccination would be an advantageous prophylactic or treatment.

Antigens can be any type of biologic molecule including, for example, simple intermediary metabolites, sugars, lipids, and hormones as well as macromolecules such as complex carbohydrates, phospholipids, nucleic acids and proteins. According to the invention, cells that comprise or are attached to a molecule that can elicit an immune response are also considered antigens. Common categories of antigens include, but are not limited to, viral antigens, bacterial antigens, fungal antigens, protozoal and other parasitic antigens, tumor antigens, antigens involved in autoimmune disease, allergy and graft rejection, and other miscellaneous antigens. In certain embodiments, vaccines of the invention comprise one or more antigens selected from the group consisting of (a) live, heat killed, or chemically attenuated viruses, bacteria, mycoplasmas, fungi, and protozoa; (b) fragments, extracts, subunits, metabolites and recombinant constructs of (a); (c) fragments, subunits, metabolites and recombinant constructs of mammalian proteins and glycoproteins; (d) tumor-specific antigens, (e) allergens, and (f) nucleic acids.

Examples of viral antigens include, but are not limited to, live, attenuated or killed forms of the following viruses or molecular components of the viruses: Rotavirus, Influenza, Parainfluenza, Herpes species, Herpes simplex, Epstein Barr Virus, Chicken Pox, Pseudorabies, Cytomegalovirus, Rabies, Polio, Hepatitis A, Hepatitis B, Hepatitis C, Hepatitis E, Measles, Distemper, Venezuelan Equine Encephalomyelitis, Feline Leukemia Virus, Reovirus, Respiratory Sycytial Virus, Lassa Fever Virus, Polyoma Tumor Virus, Canine Parvovirus, Papilloma Virus, Tick Borne Encephalitis, Rinderpest, Human Rhinovirus Species, Enterovirus Species, Mengo Virus, Paramyxovirus, Avian Infectious Bronchitis Virus, HTLV 1, HIV-1, HIV-2, Influenza A and B, LCMV (Lymphocytic Choriomeningitis Virus), Parovirus, Adenovirus, Togavirus (Rubella, Yellow Fever, Dengue Fever), Bovine Respiratory Syncicial Virus, and Corona Virus.

Bacterial antigens include the following bacteria and molecular components thereof: Bordetella pertussis, Brucella abortis, Escherichia coli, Salmonella species, Salmonella typhi, Streptococci, Vibrio (V. cholera, V. parahaemolyticus), Shigella pseudomonas, Brucella species, Mycobacteria species (tuberculosis, avium, bcg, leprosy), Pneumococci, Staphlylococci, Enterobacter species, Rochalimaia, Henselae, Pasterurella (P. haemolytica, P. multocida), Chlamydia (C. trachomatis, C. psittaci, Lymphogranuloma venereum), Syphilis (Treponema pallidum), Haemophilus species, Mycoplasmosis, Lyme disease (Borrelia burgdorferi), Botulism (Clostridium botulinum), Corynebacterium, Diphtheriae, Versinia, and Entercolitica. Additional bacterial antigens are pertussis bacterial antigens such as pertussis toxin, filamentous hemagglutinin, pertactin, FIM2, FIM3, adenylate cyclase and other pertussis bacterial antigen components; diptheria bacterial antigens such as diptheria toxin or toxoid and other diptheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components, Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components; pneumococcal bacterial antigens such as pneumolysin, pneumococcal capsular polysaccharides and other pneumococcal bacterial antigen components; Haemophilus influenza bacterial antigens such as capsular polysaccharides and other haemophilus influenza bacterial antigen components; anthrax bacterial antigens such as anthrax protective antigen and other anthrax bacterial antigen components; rickettsiae bacterial antigens such as rompA and other rickettsiae bacterial antigen component.

Fungal antigens include Candida fungal antigen components; Histoplasma fungal antigens such as heat shock protein 60 (HSP60) and other Histoplasma fungal antigen components; Cryptococcal fungal antigens such as capsular polysaccharides and other Cryptococcal fungal antigen components; Coccidiodes fungal antigens such as spherule antigens and other Coccidiodes fungal antigen components; and Tinea fungal antigens such as Trichophytin and other Coccidiodes fungal antigen components.

Protozoal and other parasitic antigens include Plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; Leishmania major and other Leishmaniae antigens such as gp63, lipophosphoglycan and its associated protein and other Leishmanial antigen components; and Trypanosoma cruzi antigens such as the 75-77 kDa antigen, the 56 kDa antigen and other trypanosomal antigen components.

Tumor antigens include telomerase; multidrug resistance proteins such as P-glycoprotein; MAGE-1, alpha fetoprotein, carcinoembryonic antigen, mutant p53, Papillomavirus antigens, gangliosides or other carbohydrate-containing components of melanoma or other tumor cells. It is contemplated by the invention that antigens from any type of tumor cell can be used in the compositions and methods described herein.

Antigens involved in autoimmune diseases, allergy, and graft rejection also can be used in the compositions and methods of the invention. For example, an antigen involved in any one or more of the following autoimmune diseases or disorders can be used in the present invention: diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, and neural graft components. The antigen may be an altered peptide ligand useful in treating an autoimmune disease.

Vaccines of the invention may further contain an adjuvant other than the anti-microtubule agent, to further boost the stimulated immune response. The additional adjuvant may be any non-immunogenic compound that, when administered with an antigen, enhances or modifies the immune response to that particular antigen. The additional adjuvant may be any of those already known and described. For example, the adjuvant may be an aluminum compound, an oil-in-water emulsion, Freund's adjuvant, a pertussis adjuvant, a muramyl peptide or a saponin.

The vaccine compositions, including (i) an antigen and (ii) anti-microtubule agent, are usefully employed to induce an immunological response in an animal, by administering to such animal an effective amount of the vaccine composition. The term “effective amount” refers to an amount sufficient to enhance a host defense mechanism. This amount may vary to some degree depending on the mode of administration, but will be in the same general range. The exact effective amount necessary could vary from recipient to recipient, depending on the species, age and general condition of the recipient, the relevant disease condition, the mode of administration, and so forth. Thus, it is not possible to specify an exact effective amount. However, the appropriate effective amount may be determined by one of ordinary skill in the art using only routine experimentation or prior knowledge in the vaccine art.

Appropriate modes for administering compositions of the present invention include parenteral administration, such as subcutaneous (SC) injection, transcutaneous, intranasal (IN), ophthalmic, transdermal, intramuscular (IM), intradermal (ID), intraperitoneal (IP), intravaginal, pulmonary, and rectal administration, as well as non-parenteral administration, such as oral administration and inhalation.

Compositions of the invention may be formulated with other constituents that do not unduly interfere with the immune-stimulating quality of the compositions. This may be accomplished according to conventional pharmaceutical techniques. See, for example, Remington's Pharmaceutical Sciences, 17th Ed. (1985, Mack Publishing Co., Easton, Pa.). Typically, the active ingredients will be admixed with one or more pharmaceutically acceptable carriers, a term that refers a carrier that does not cause an allergic reaction or other untoward effect in recipients. The carrier may take a wide variety of forms, depending on the form of preparation desired for administration. The compositions may further contain antioxidizing agents, stabilizing agents, dispersing agents, preservatives and the like.

For parenteral administration, active agents may be dissolved in or mixed with a pharmaceutically acceptable carrier. Illustrative of suitable carriers are water, saline, dextrose solutions, fructose solutions, ethanol, or oils of animal, vegetative or synthetic origin. The compositions may also contain other ingredients, for example, preservatives, suspending agents, dispersing agents, solubilizing agents, buffers and the like. Formulations for parenteral administration may be presented in unit dosage form, e.g., in ampules or vials, or in multi-dose containers, with or without an added preservative. The composition can be formulated as a solution, a suspension, or an emulsion in oily or aqueous vehicles. Alternatively, compositions may be in lyophilized powder form, for reconstitution with a suitable vehicle, e.g., sterile, pyrogen-free water or physiological saline.

For oral administration, compounds can be formulated into solid or liquid preparations such as capsules, pills, tablets, lozenges, melts, powders, suspensions or emulsions. In preparing compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract.

The compositions can be given in a single dose schedule or in a multiple dose schedule. A multiple dose schedule is one in which a primary course of vaccination can include 1-10 separate doses, followed by other doses given at subsequent time intervals required to maintain and or reinforce the immune response, for example, at 1-4 months for a second dose, and if needed, a subsequent dose(s) after several months. Periodic boosters at intervals of 1-5 years, usually 3 years, may be desirable to maintain the desired levels of protective immunity. The course of the immunization can be followed by in vitro assays.

The following examples are intended to illustrate and provide a more complete understanding of the invention without limiting the invention to the examples provided.

### EXAMPLE 1

### A High Throughput Screening Method for Identifying Agents that Stimulate the Innate Immune System

This example shows a high throughput screening method useful for identifying agents that stimulate the innate immune system.

Materials and Methods

Cell Culture

Human monocytic cell lines THP-1 (TIB-202; ATCC) were grown in RPMI-1640 media (Cambrex) supplemented with 10% FCS (HyClone), 2 mM L-glutamine (Sigma-Aldrich), 50 μM 2-mercaptoethanol (Sigma-Aldrich), and sodium pyruvate (Invitrogen).

Test Compounds Preparation and Storage

Test compounds/candidate agents were diluted in 100% DMSO at a concentration of 10⁻²M and stored in 96 well “matrix” plates at −80° C. These compound stocks were employed as a pool of mother plates. Compounds to be assayed were diluted 100 times in sterile PBS using a liquid handling robot “Evolution P3” (PerkinElmer) to a concentration of 10⁻⁴M, and stored at −20° C. until they were used for the assay. In total, 20,000 candidate agents were tested.

HTS Assay Format

10⁵ THP-1 cells were incubated overnight with 10⁻⁵M of test compound (20 μl of 10⁻⁴M compound stock solution was added to 180 μl of the cells suspension). Incubations were carried out in U bottom 96 well tissue culture plates. Each compound was tested in duplicate. Collection of the cell culture supernatants and cell surface receptor staining were carried out using the “Biomek 2000” liquid handling robot (Becton Coulter).

Flow Cytometry

Expression of cell surface receptors was analyzed by flow cytometry using the following antibodies: PE-conjugated anti-human CD80 and APC conjugated anti-human CD54 (ICAM-1) and matching labeled isotype controls, all from BD Pharmingen. Cells in the first well of each duplicate were stained with isotype controls (IC). In the second well, cells were stained with CD80 and CD54 antibodies. Stained cells were analyzed on a flow cytometer (FACSArray cytometer (BD Pharmingen)). The mean fluorescence intensity (MFI) for IC and CD80/CD54 antibodies stained cells were determined using “FlowJo” software (Tree Star, Inc.). The IC MFIs were subtracted from CD80/CD54 MFIs and results were recorded as specific staining and used for the following data analysis.

Cytokine/Chemokine Detection

The concentrations of IL-8, RANTES and TNFα in cell culture supernatants were determined for each well of the duplicates using the “Fluorokine MultiAnalyte Profiling” kits (R&D Systems). Samples were analyzed on the Luminex 100IS system and data analysis was performed using Luminex 2.3IS software (both from Luminex corporation). The mean value of two wells was recorded and used for the following data analysis.

Results

TABLE 1
Data demonstrating immunomodulatory activity of the Colchicine,
Vinblastine, Vincristine and Demecolcine.

| Name | CD54 | CD80 | IL-8 | RANTES | TNFα |
|---|---|---|---|---|---|
| Negative Control | 26.71 | 0.49 | 42.98 | 228.09 | 9.24 |
| (vehicle only) | | | | | |
| Positive Control | 3662.56 | 64.12 | 13562.99 | 3217.60 | 866.78 |
| (LPS) | | | | | |
| Colchicine | 554.50 | 3.69 | 7499.42 | 4383.62 | 66.37 |
| Vinblastine | 665.18 | 3.89 | 6543.63 | 4568.01 | 78.47 |
| Vincristine | 814.96 | 3.90 | 8334.51 | 5232.60 | 88.30 |
| Demecolcine | 600.87 | 4.91 | 2533.58 | 740.62 | 95.20 |

To identify agents that stimulate the innate immune system, a multiplex functional cell-based assay was used. The assay used human monocytes (THP-1 cell line) as target cells and expression of co-stimulatory molecules (CD54 and CD80) and immune-activating cytokines (IL-8, RANTES and TNFα) as assay readouts. These molecules play major role in the innate immune response and are required for effective activation of the adaptive immune system. Compounds that showed activity in the assay were predicted to possess potent immune-stimulating properties.

The assay background level was established using cells incubated with the compound's diluent only, and the level of maxim cellular response was determined by incubating cells with potent activator of the innate immune system bacterial lipopolisaccharide (LPS), as shown in Table 1.

Incubation of the THP-1 cells with microtubule de-polymerizing compounds resulted in significant expression up-regulation of four out of five proteins used as the assay readouts: CD54 (ICAM-1), IL-8, RANTES and TNFα (see Table 1). The data demonstrated that tested microtubule de-polymerizing compounds are potent activators of the innate immune system, and indicated that those compounds can be used either as nonspecific activators of an innate immune response or as potent adjuvants for new vaccines.

### EXAMPLE 2

### Determination of Toll-Like Receptor Expression on Cells

This example shows that RT-PCR can be used to determine whether a cell expresses a TLR.

PCR primers for specific for TLR-1, TLR-2, TLR-3, TLR-4, TLR-5, TLR-6, TLR-7, TLR-8, TLR-9, and TLR-10 were designed using known nucleic acid sequences encoding those receptors. The primers were used according to standard RT-PCR protocols to amplify mRNA transcripts in three cell lines: THP-1, HL-60, and HEK-TLR9. β-actin was used as a positive control in the RT-PCR. Electrophoresis was performed on all RT-PCR products.

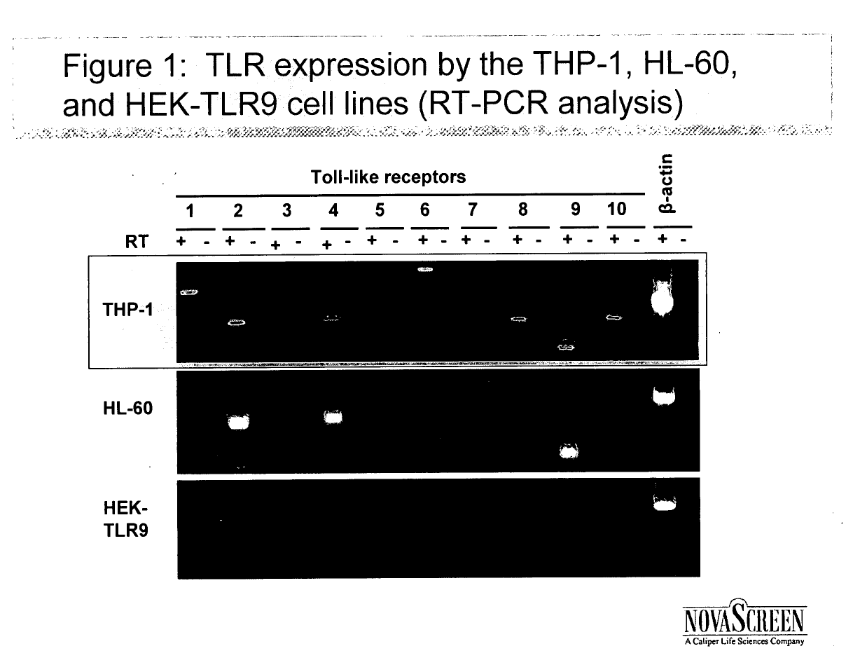

Results are shown in FIG. 1. THP-1 was shown to express significant quantities of TLR-1, TLR-2, TLR-4, TLR-6, TLR-7, TLR-8, TLR-9 and TLR-10. HL-60 was shown to express significant quantities of TLR-2, TLR-4, TLR-6, TLR-7, and TLR-9. HEK-TLR9 was shown to express significant quantities of TLR-9 only.

### EXAMPLE 3

### Demonstration that Ligand Binding to TLRs on THP-1 Cells Upregulates Expression of Cell Surface Molecules Involved in Innate Immunity

This example shows that ligand binding to TLRs on THP-1 cells upregulates the expression of cell surface molecules involved in innate immunity.

THP-1 cells were incubated with IFN-γ, LPS or a control. Flow cytometry analysis was used to determine how binding of IFN-γ and LPS to TLRs affected the expression of cell surface molecules involved in innate immunity.

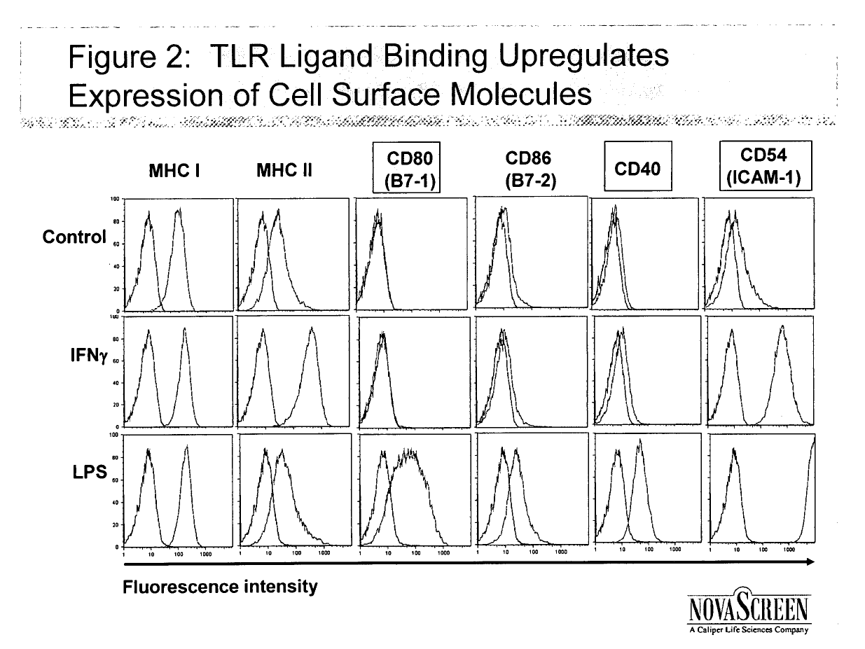

Results are shown in FIG. 2. Ligand binding upregulated the surface expression of MHC class I, MHC class II, CD80 (B7-1), CD40, and CD54 (ICAM-1). Ligand binding did not significantly affect the surface expression of CD86 (B7-2).

### EXAMPLE 4

### Demonstration that Ligand Binding to TLRs on THP-1 Cells Upregulates Expression of Cytokines Involved in Innate Immunity

This example shows that ligand binding to TLRs on THP-1 cells upregulates the expression of cytokines involved in innate immunity.

THP-1 cells were incubated with FSL-1, PAM2, PAM3, poly IC, LPS, Flagellin, Resquimod, E. coli DNA, or a control. Commercially available cytokine detection kits were used to determine how binding of these ligands to TLRs affected the expression of cytokines.

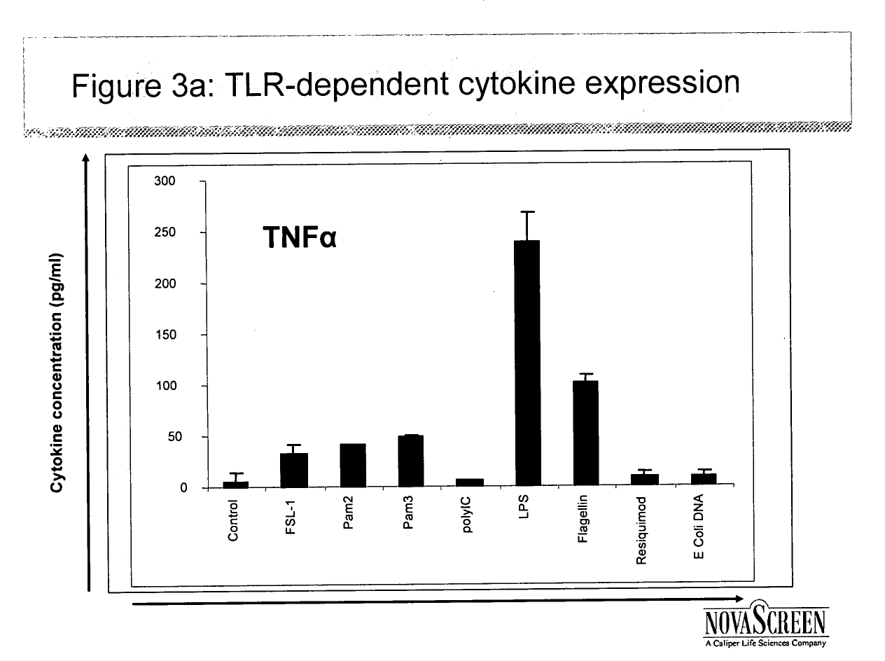

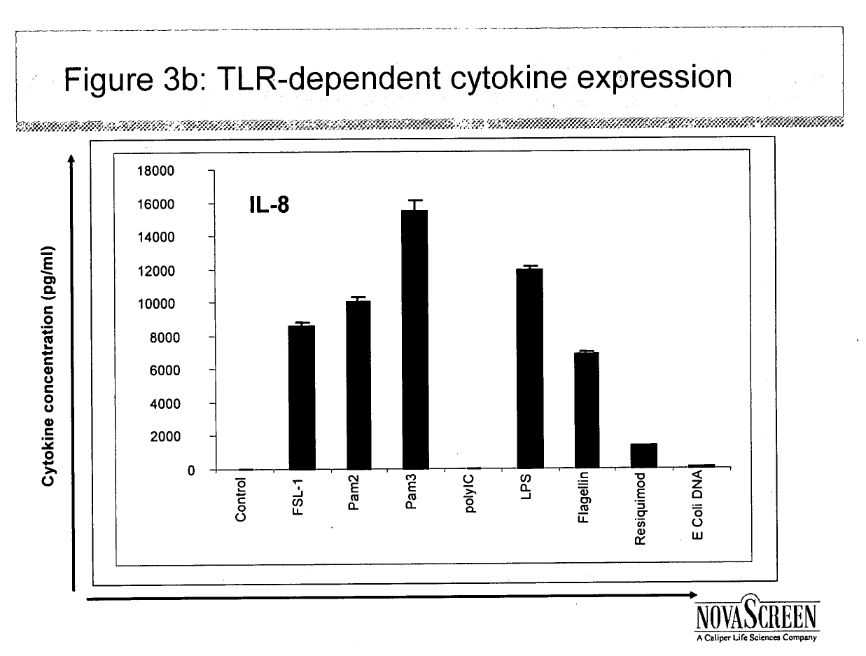

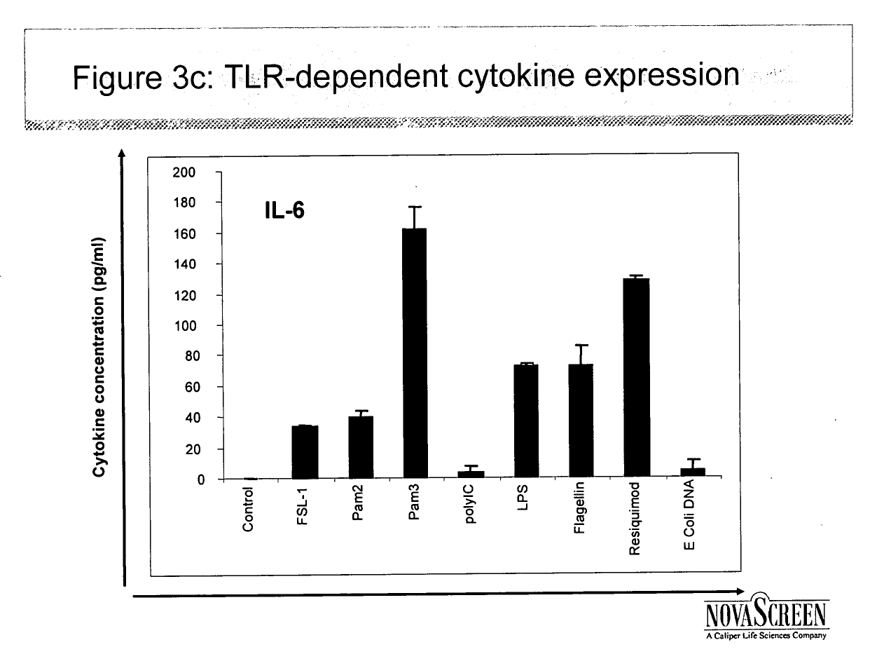

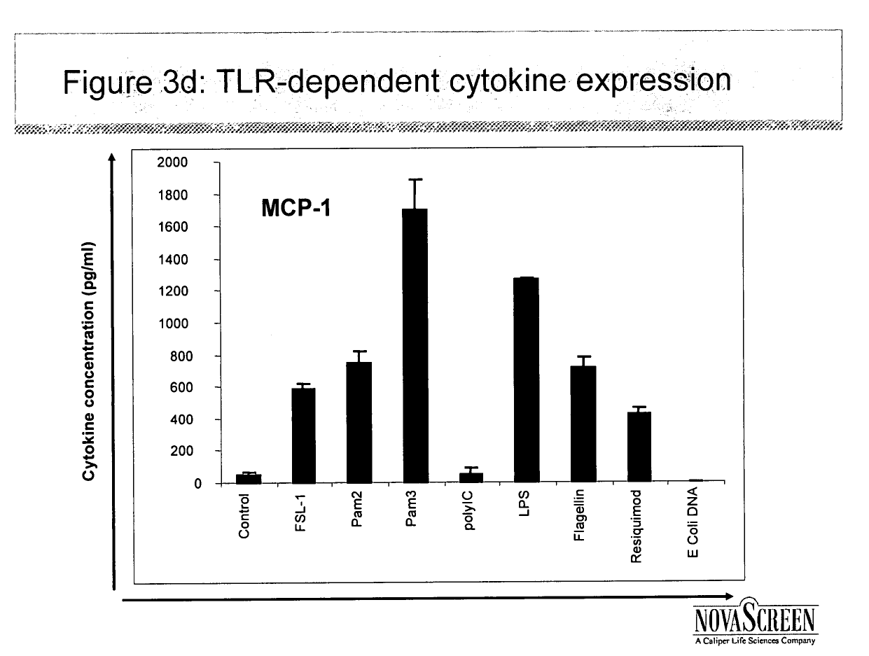

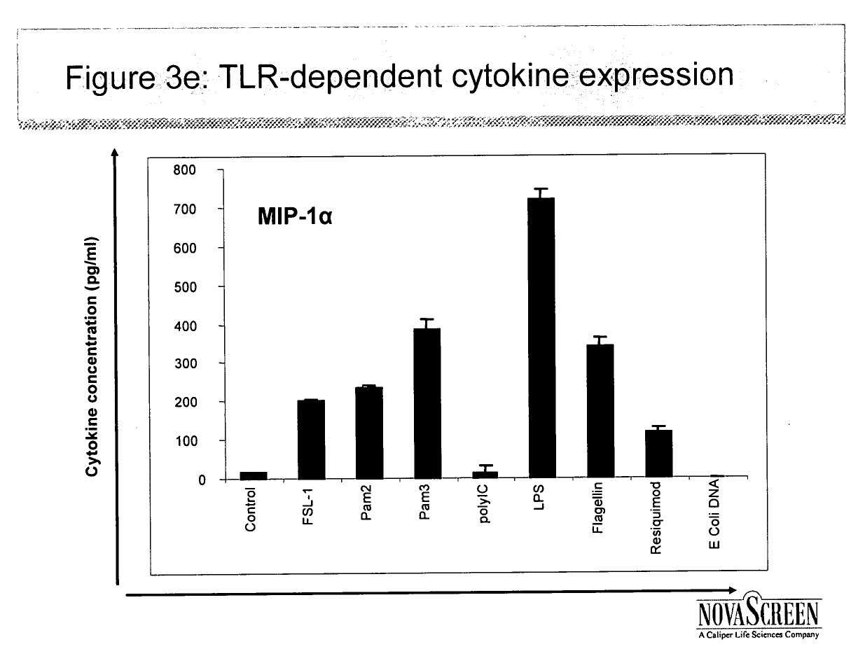

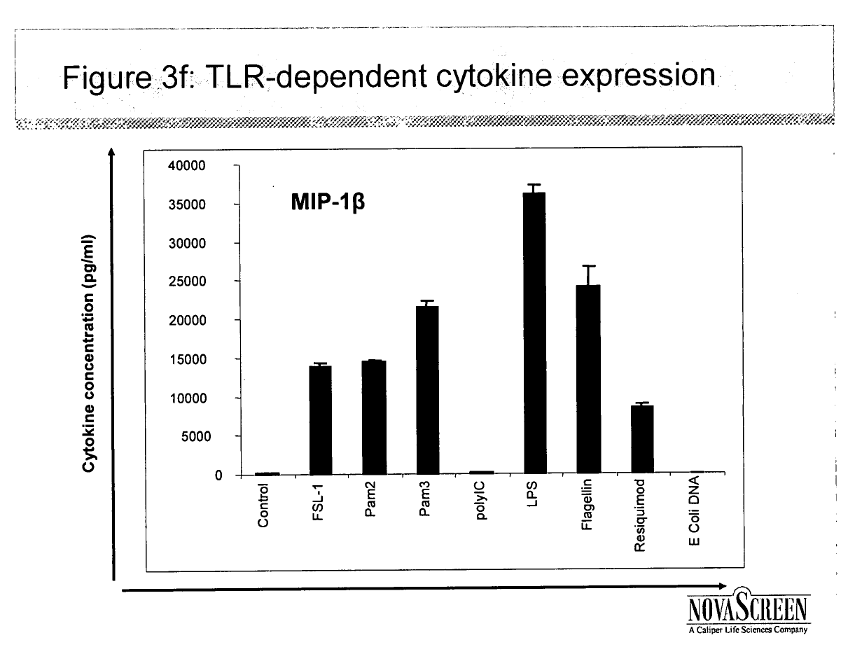

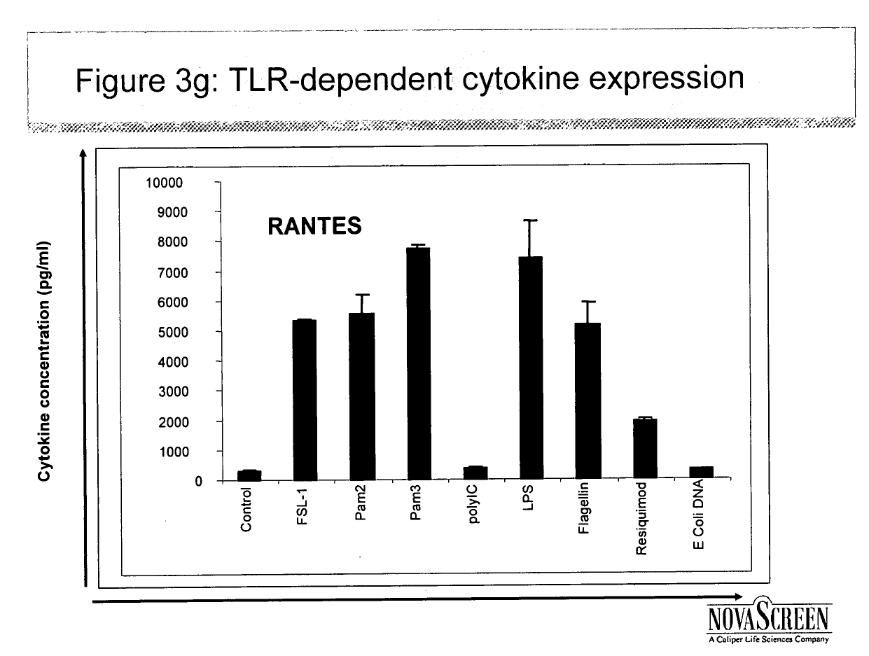

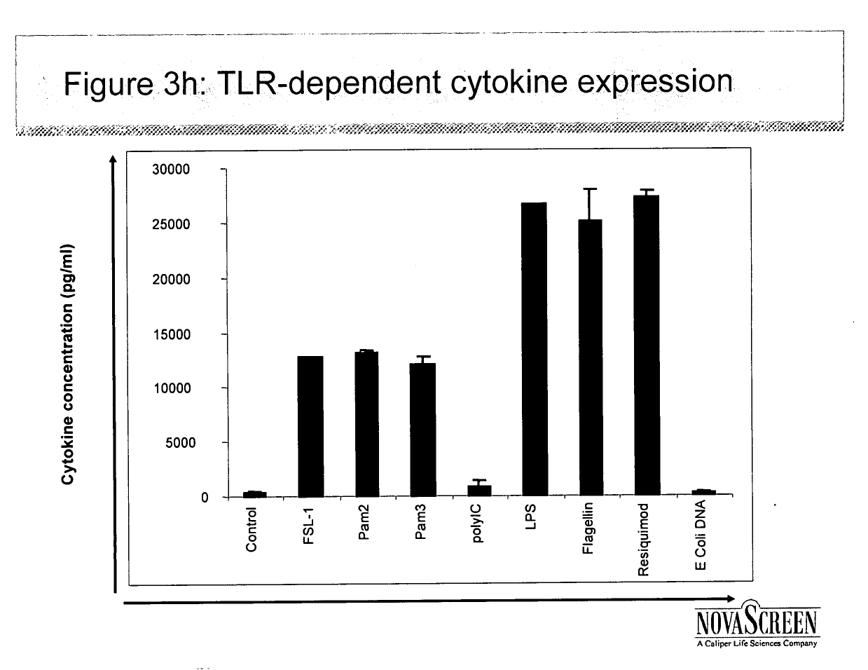

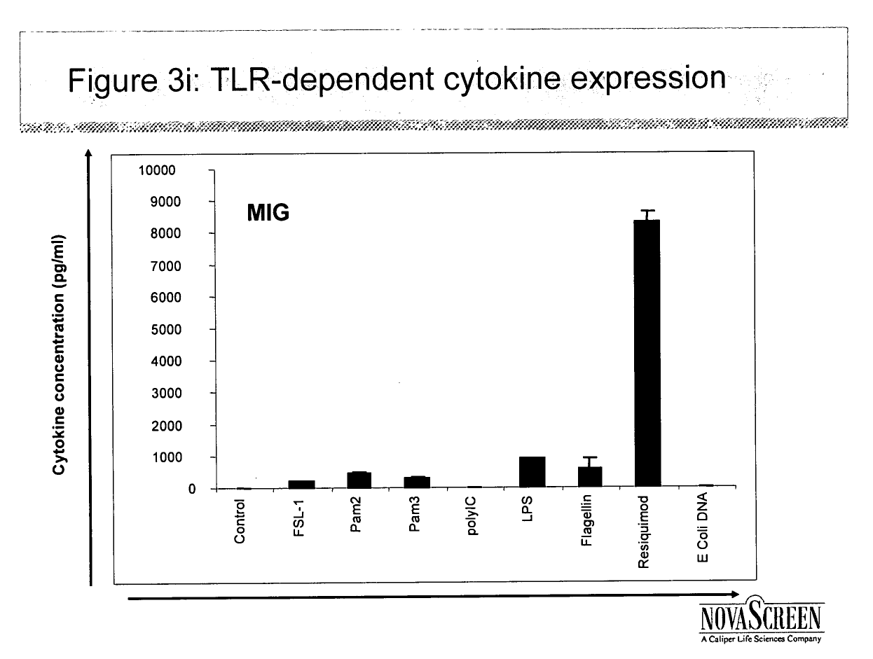

Results are shown in FIG. 3. Ligand binding variously upregulated the expression of TNF-α, IL-8, IL-6, MCP-1, MIP-1 α, MIP-1β, RANTES, IP-10 and MIG.

### EXAMPLE 5

### Demonstration of Selecting a Panel of Markers for Use in a Screening Assay for Identifying Agents that Stimulate the Innate Immune System

This example demonstrates the selection of a panel of markers used to screen for agents that stimulate the innate immune system.

THP-1 cells were incubated with FSL-1, PAM2, PAM3, poly IC, LPS, Flagellin, Resquimod, E. coli DNA, or a control. The levels of cell surface molecules and cytokines involved in innate immunity were then measured, using the methods described in previous examples, as an indicator of the effect of ligand binding to TLRs on the THP-1 cells.

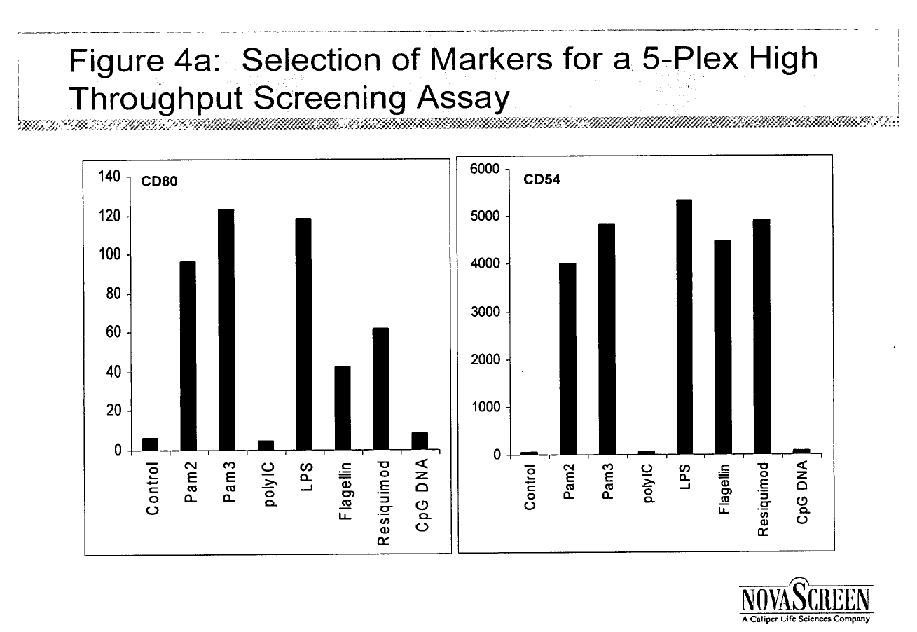

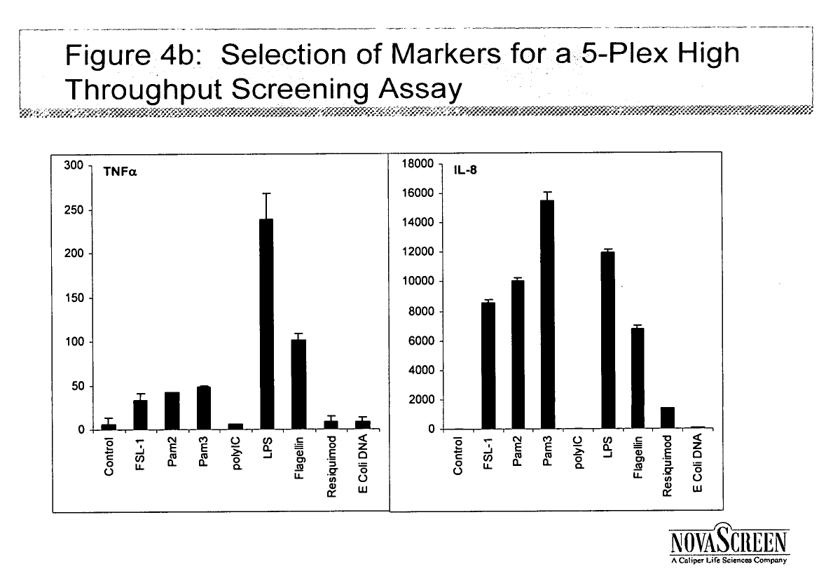

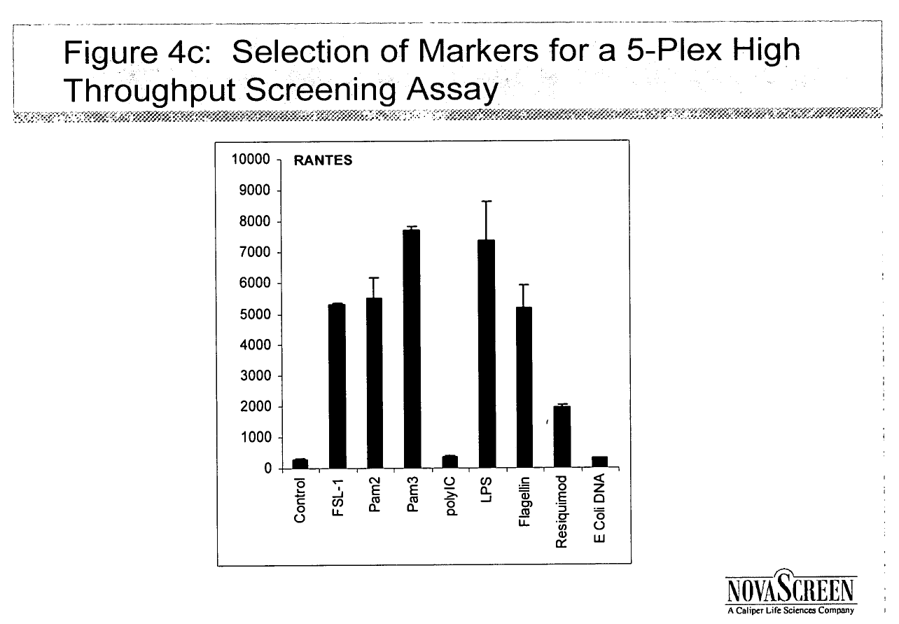

Results are shown in FIG. 4. Ligand binding significantly upregulated CD80, CD54, TNF-α, IL-8 and RANTES, all of which were selected to be used in a panel of markers used for high throughput screening.

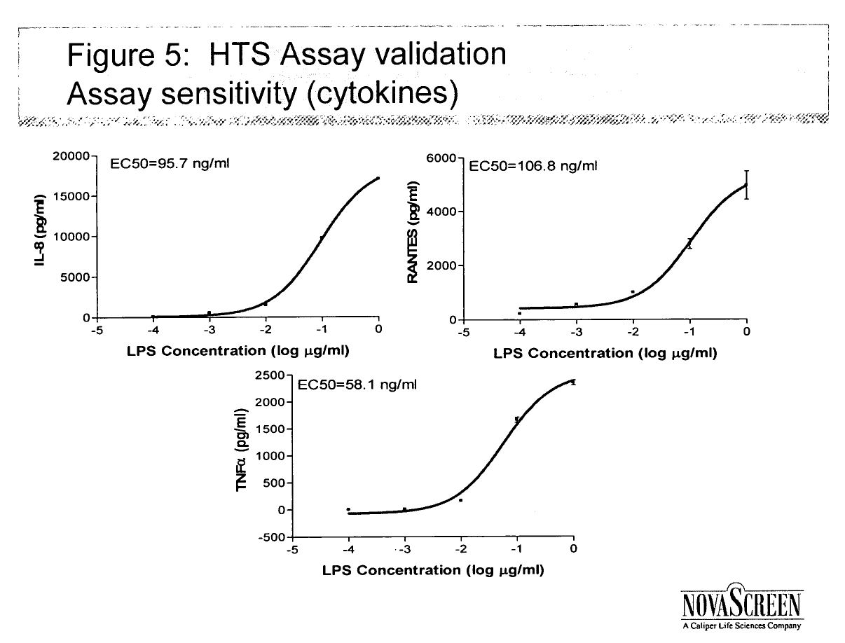

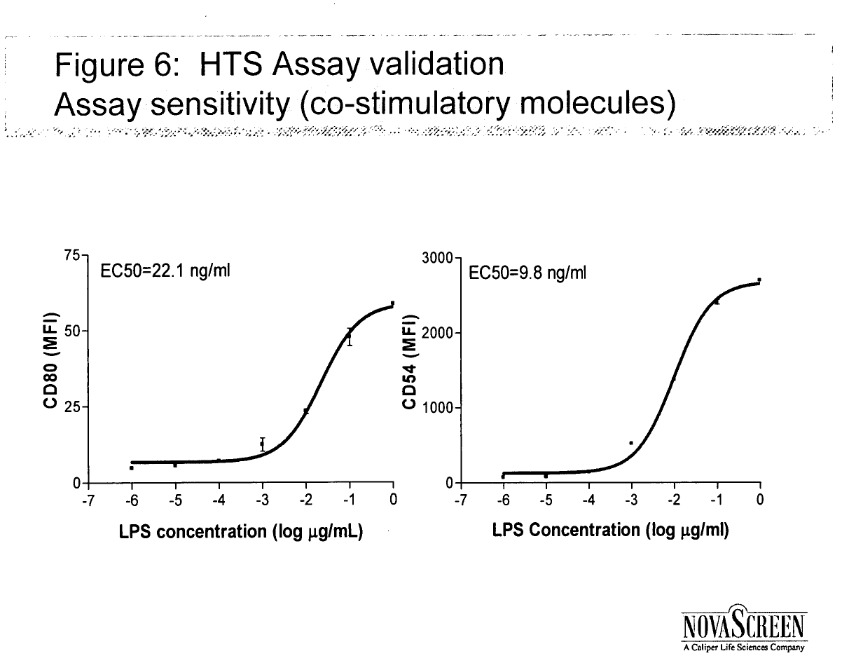

Sensitivity of the assays was determined for each cytokine and cell surface marker in response to LPS binding to TLRs. For this experiment THP-1 cells were treated with LPS at a range of concentrations varying from 1 μg/ml to 0.01 ng/ml. Concentration of IL-8, RANTES and TNFα in cell culture supernatants was measured using the Luminex technology. Expression of the CD54 and CD80 were determined by flow cytometry using the FACSArray counter. The results were plotted and used for the calculation of EC50 values (the point at which 50% of maximum effect is observed) for each of the five assay readouts. Results for the cytokines are shown in FIG. 5. Results for the cell surface markers (co-stimulatory molecules) are shown in FIG. 6.

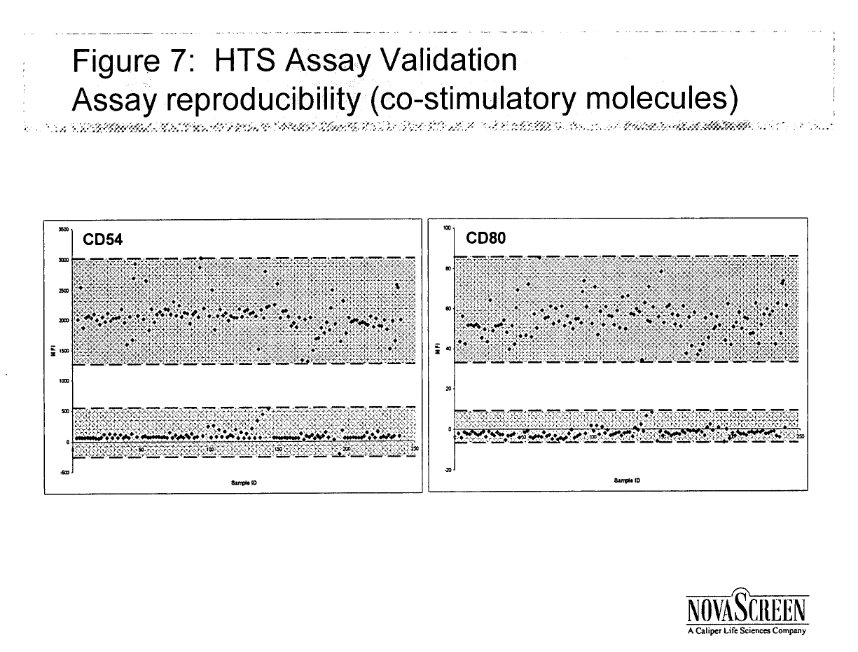

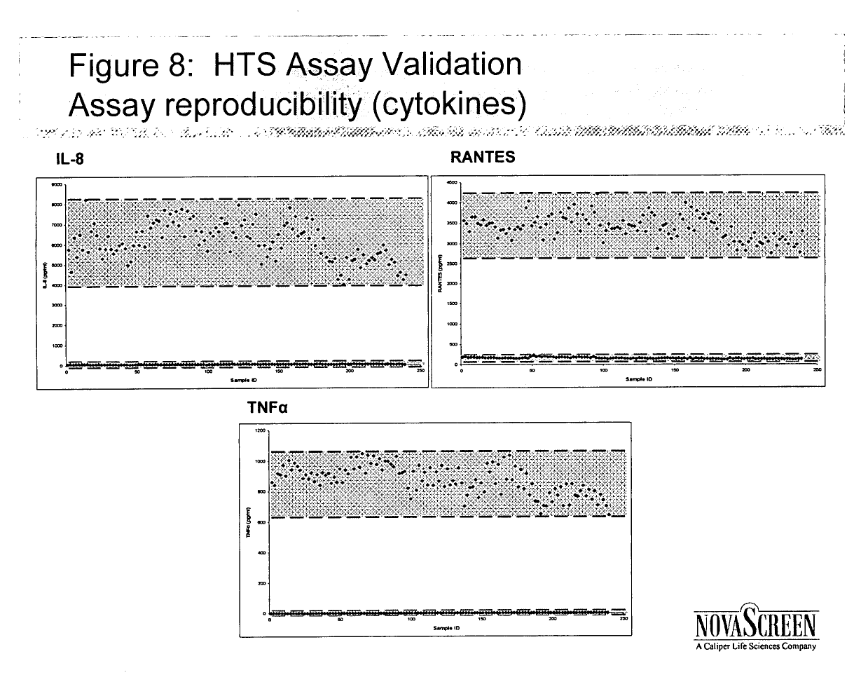

To validate assay reproducibility a series of multi-plate experiments consisting of sequences of control/sample wells across each plate were set up. In control wells, cells were incubated with media only. In the sample wells, cells were stimulated with LPS (TLR4 agonist). Cell cultures were then analyzed for expression of CD54, CD80, IL-8, RANTES and TNFα Results are shown in FIGS. 7-8. Data from these experiments were used to calculate a Z′ factor value (Table 1). The Z′ values for all five assay readouts routinely exceeded the 0.5 cut-off point which is indicative of acceptable performance in HTS assays.

TABLE
Z′ factor values for each component
of the 5-plex HTS assay system.

| | CD54 | CD80 | IL-8 | RANTES | TNFα |
|---|---|---|---|---|---|
| Plate 1 | 0.67 | 0.64 | 0.59 | 0.69 | 0.79 |
| Plate 2 | 0.73 | 0.50 | 0.68 | 0.72 | 0.82 |
| Plate 3 | 0.55 | 0.47 | 0.55 | 0.70 | 0.68 |
| Plate 4 | 0.64 | 0.47 | 0.54 | 0.68 | 0.63 |
| Plate 5 | 0.66 | 0.59 | 0.63 | 0.78 | 0.75 |
| Averaqe | 0.65 | 0.53 | 0.60 | 0.72 | 0.73 |
| SD | 0.06 | 0.08 | 0.06 | 0.04 | 0.08 |

What is claimed is:

1. A method of screening for agents that stimulate the innate immune system in a mammal, comprising:
(a) bringing a candidate agent into contact with a cellular component of said innate immune system,
(b) testing said cellular component for the level of one or more markers associated with stimulation of said innate immune system, and
(c) correlating said level with a probability that said candidate agent stimulates said innate immune system.

2. The method according to claim 1, wherein said cellular component is selected from the list consisting of a monocyte, a dendritic cell, a macrophage, a B-cell and a natural killer cell.

3. The method according to claim 2, wherein said cellular component is a monocyte.

4. The method according to claim 3, wherein said cellular component is a THP-1 cell.

5. The method according to claim 1, wherein said marker is an antigen presenting molecule of the major histocompatibility complex.

6. The method according to claim 1, wherein said marker is a costimulatory molecule.

7. The method according to claim 6, wherein said costimulatory molecule is selected from the group consisting of CD80 (B7-1), CD40 and CD54 (ICAM-1).

8. The method according to claim 1, wherein said marker is a cytokine.

9. The method according to claim 8, wherein said cytokine is selected from the group consisting of TNFA, IL-8, IL-6, MCP-1, MIP-1αMIP-1β, RANTES, IP-10 and MIG.

10. The method according to claim 9, wherein said cytokine is selected from the group consisting of TNFα, IL-8, and RANTES.

11. The method according to claim 1, wherein said mammal is a human.

12. The method according to claim 1, wherein said one or more markers is a panel of markers.

13. The method according to claim 12, wherein said panel of markers comprises, CD80, CD54, IL-8, RANTES, and TNFα.

14. A method of stimulating the innate immune system in a mammal, comprising administering a vaccine and a microtubule depolymerizing agent to said mammal.

15. The method according to claim 14, wherein said microtubule depolymerizing agent is selected from the list consisting of colchicine, vinblastine, vincristine, and demecolcine.

16. The method according to claim 14, wherein said microtubule depolymerizing agent is a component of said vaccine.

17. The method according to claim 14, wherein said microtubule depolymerizing agent is administered prior to said vaccine.

18. The method according to claim 14, wherein said microtubule depolymerizing agent is administered subsequent to said vaccine.

19. The method according to claim 14, wherein said microtubule depolymerizing agent is administered concurrently with said vaccine.

20. The method according to claim 14, wherein said mammal is a human.

21. A method of stimulating the innate immune system in a mammal, comprising:
(a) selecting a mammal in need of increased innate immunity that does not have a cell proliferative disorder, and
(b) administering a microtubule depolymerizing agent to said mammal.

22. The method according to claim 21, wherein said microtubule depolymerizing agent is selected from the list consisting of colchicine, vinblastine, vincristine, and demecolcine.

23. The method according to claim 21, wherein said cell proliferative disorder is cancer.

24. The method according to claim 21, wherein said mammal is a human.

25. A vaccine that comprises a microtubule depolymerizing agent.

26. The vaccine of claim 28, wherein said microtubule depolymerizing agent is selected from the list consisting of colchicine, vinblastine, vincristine, and demecolcine.
